(19) United States
(12) Patent Application Publication
(10) Pub. No.: US 2022/0151955 A1
(43) Pub. Date: May 19, 2022

(54) KETAMINE FOR THE TREATMENT OF POSTPARTUM SYMPTOMS AND DISORDERS

(71) Applicant: The Ketamine Research Foundation, San Anselmo, CA (US)

(72) Inventors: Philip Elihu WOLFSON, San Anselmo, CA (US); Rob COLE, San Anselmo, CA (US); Melissa WHIPPO, San Anselmo, CA (US); Julane ANDRIES, San Anselmo, CA (US)

(21) Appl. No.: 17/431,468

(22) PCT Filed: Aug. 5, 2020
(86) PCT No.: PCT/US2020/045005

§ 371 Date: Aug. 17, 2021

Related U.S. Application Data
(60) Provisional application No. 62/882,858, filed on Aug. 5, 2019.

(51) Int. Cl. A61K 31/135; A61K 45/06; A61P 25/24; A61P 25/22

(52) U.S. Cl. CPC A61K 31/135; A61P 25/22; A61P 25/24; A61K 45/06

(57) ABSTRACT

The present invention features methods, compositions, and kits for the treatment of all forms of emotional I symptoms related to the postpartum period, however so defined in terms of the length of the period following delivery, including symptoms characteristic of postpartum difficulties including RPR, PPD, PPM, the baby blues, and sub-diagnostic emotional, psychological, cognitive, spiritual, and/or physical symptoms related to the postpartum period.

This application claims the benefit of U.S. Provisional Ser. No. 62/882,858, filed Aug. 5, 2019, which is incorporated herein by reference.

### BACKGROUND OF THE INVENTION

The present invention features methods, compositions, and kits for the treatment of all forms of emotional and psychological symptoms and disorders related to childbirth and its aftermath, including symptoms characteristic of Postpartum Mood and Anxiety Disorders (PMAD), Postpartum Psychosis (PPP), Postpartum Depression (PPD), Anxiety (PPA), Post-Traumatic Stress (PPPTSD), Obsessive Compulsive Disorder (PPOCD) and Postpartum Bipolar I and II disorders (PPBPD) sub-diagnostic emotional, psychological, cognitive, spiritual, and/or physical symptoms of the postpartum period, as well as recrudescence and relapse of disorders and emotional difficulties from pre-postpartum life .

Postpartum Mood and Anxiety Disorders (PMAD) impact 20% of women. They are the number one complication of the postpartum setting. Women of every culture, age, income level and race can develop perinatal mood and anxiety disorders. Symptoms can appear any time during pregnancy and the first 12 months after childbirth. The American College of Obstetrics and Gynecology (ACOG) recommends women obtain screening for PMAD.

PMAD encompasses a range of mood disorders, including depression, anxiety, bipolar phases, obsessive compulsive, post-traumatic stress, and also psychosis. Postpartum psychosis (PPP) is rare, impacting only 1% of new mothers, but it is dangerous to mothers and newborns and is an imperative for immediate treatment. Women typically exhibit PPP symptoms, in the first few days following the baby's birth. The other disorders, such as depression (PPD), anxiety (PPA), post-traumatic stress (PPPTSD), obsessive compulsive disorder (PPOCD) and bipolar (PPBPD), typically show themselves after the initial two weeks postpartum and at any time throughout the first 12 months following childbirth. Ketamine's many decades long use has established its safety. In this method dosages are much lower than that used for anesthesia and side effects are minimal.

During the first two weeks postpartum, 80-90% of women experience the “baby blues.” Symptoms of the baby blues can look like depression and anxiety and include crying, mood liability and quickness to anger. These symptoms are attributed to hormonal changes in the body following childbirth, lack of sleep and the overall adjustment to new motherhood. Clinical intervention may not be needed for these symptoms. Some women and their partners and families may wish for and benefit from supportive interventions that reduce the attendant anxiety and give vent to the In general, it is recommended for all new mothers and their immediate network have as much practical support as possible as they adjust to the rigors of baby-care.

Any complications in childbirth or feeding following delivery may also contribute to PMAD. Complex psychosocial stressors, such as poor housing, IPV and economic stress put some women at increased risk for PMAD. Women with any history of mood disorders, however remote, are also at increased risk for PMAD. While the research remains inconclusive regarding the cause of PMAD, the best working hypothesis is that it's not merely the changing hormones in a woman's body that causes PMAD, rather it appears to be the way that these hormonal changes impact a woman's brain chemistry. Research demonstrates that treatment, whether with SSRIs, psychotherapy, support groups or a combination of all these modalities, can help women recover from PMAD. Practical support is also indicated when women suffer from PMADs, including postpartum doulas, lactation consultants and in home support, as well as facilitating sleep.

Recently released by the FDA is the first drug specifically indicated for the treatment of postpartum depression, called brexanolone (brand name Zulresso). Unfortunately, it has many drawbacks to its use including interruption of breast feeding. It must be given in a health care facility under medical supervision and is delivered intravenously over the course of 60 hours (2.5 days). Studies of brexanolone found that the drug had quick and effective results. Two clinical trials of about 250 women with postpartum depression showed that within 60 hours, 50 percent of the women who received brexanolone were no longer clinically depressed, compared with 25 percent of women who received a placebo. Brexanolone works in a different way than other drugs for depression, as it binds to GABA receptors. Yet, it is still unclear how brexanolone works Because some women who received brexanolone experienced excessive sedation and sudden loss of consciousness, the drug needs to be administered under supervision—so another handicap to its use.

Treatment for PMAD has included psychotherapy, medication (typically SSRIs and other antidepressants) and more recently the addition of mindfulness practices, acupuncture and yoga. The disparity of treatment options shows itself most strongly across socioeconomic lines. Women who have private insurance have more access to psychiatric care, support groups and individual psychotherapy. Women living at or below the poverty line and have public insurance are much more limited in their access to treatment options. Ketamine could be a treatment that crosses socioeconomic lines and potentially have less of a stigma and side effects than SSRIs.

In communities of color, it is widely reported that men hold the belief that SSRIs are “addictive” or that pregnant women and new mothers should not be “taking pills.” KAP could offer a more palatable solution as it will mostly take place in a clinic or psychiatric office setting with supervised medical care, which is less stigmatized, and not an everyday medication. As ketamine's psychiatric research continues and with it becoming a first-line treatment for depression, insurance companies will likely cover more costs and make this treatment option available to a wider population of those in need.

Many women who are breastfeeding are also skeptical of taking medications in the postpartum period, the effects on newborns and infants is not delineated adequately to be assuring of normal development. Prolonged everyday use is strikingly different than episodic short term use as with ketamine in its KAP format. Ketamine has a half-life of less than two and a half hours and breast feeding can be interrupted for short periods of time to reduce exposure of infants to significant amounts of ketamine, Women tend to prefer behavioral interventions that often are not enough to treat PMAD with more severe features but have strong feelings in opposition to taking psychiatric medications. The short term nature of KAP, as well as the potential for it not to interrupt breastfeeding, make KAP a more viable option when behavioral intervention is not sufficient.

In the rarer cases of PPP and acute suicidality, Ketamine as KAP could be quite effective. At this time, the only inpatient psychiatric unit for new mothers is in South Carolina. With 6 beds, it is always full. Ketamine treatment for PPP could potentially be offered in the postpartum unit of birth centers as well as in outpatient clinics and office. Treatment of PPP has generally consisted of lithium, antipsychotics and tranquilizers as the most effective combination to date.

Overall, the spectrum of post-partum emotional difficulties may include dysphoria, diminished interest or pleasure in activities, decrease or increase in appetite, troubled sleep or hypersomnia, insomnia, psychomotor agitation or retardation, fatigue or loss of energy, irritability, feelings of worthlessness or excessive or inappropriate guilt, diminished ability to think or concentrate or indecisiveness, inability to bond with the infant, anhedonia, anxiety, depression and in more severe states recurrent thoughts of death, suicidal ideation or suicidal attempts and anger and aggressive feelings and behavior, potentially directed towards the newborn, or partners and parents. A variety of somatic symptoms may also be present. These include varying degrees of fatigue, back pain, breast tenderness and discomfort, headaches and other manifestations. Underlying emotional and physical problems may be exacerbated including dysphoria, anxiety and Post-Traumatic Stress Disorder. Exacerbation or recrudescence of prior Bipolar I and II disorders and depression may occur. Women who have a prior history of these disorders are at greater risk for PPD and PPP. Difficulties between partners, with prior children, relatives and friends may become heightened and can cause alienation, fracturing and contribute to separation and divorce.

Applicants have discovered that ketamine can be useful for ameliorating postpartum symptoms and disorders, including symptoms characteristic of postpartum-related disorders, such as PMAD, PPP, PPD, PPA, PPPTSD, PPOCD, and PPBPD.

### SUMMARY OF THE INVENTION

The invention features a method of treating a postpartum related emotional symptom in a subject in need thereof, the method including administering to the subject ketamine, norketamine, 6-hydroxynorketamine, or a pharmaceutically acceptable salt thereof, in an amount sufficient to treat the postpartum related symptom. The postpartum related symptom can be selected from, or generalized to include dysphoria, diminished interest or pleasure in activities, decrease or increase in appetite, troubled sleep or hypersomnia, insomnia, psychomotor agitation or retardation, fatigue or loss of energy, irritability, feelings of worthlessness or excessive or inappropriate guilt, diminished ability to think or concentrate or indecisiveness, inability to bond with the infant, anhedonia, anxiety, depression and in more severe states recurrent thoughts of death, suicidal ideation or suicidal attempts and anger and aggressive feelings and behavior—potentially directed towards the newborn, or partners and parents—and a variety of somatic symptoms that may also be present which may include varying degrees of fatigue, back pain, breast tenderness and discomfort, headaches and other manifestations—as well as underlying emotional and physical problems that may be exacerbated including dysphoria, anxiety and Post-Traumatic Stress Disorder but not limited to these.

In a related aspect, the invention features a method of treating postpartum symptoms in a subject in need thereof, the method including administering to the subject ketamine, norketamine, 6-hydroxynorketamine, or a pharmaceutically acceptable salt thereof, in an amount sufficient to treat the postpartum symptoms . In particular embodiments, the method ameliorates dysphoria, diminished interest or pleasure in activities, decrease or increase in appetite, troubled sleep or hypersomnia, insomnia, psychomotor agitation or retardation, fatigue or loss of energy, irritability, feelings of worthlessness or excessive or inappropriate guilt, diminished ability to think or concentrate or indecisiveness, inability to bond with the infant, anhedonia, anxiety, depression and in more severe states recurrent thoughts of death, suicidal ideation or suicidal attempts and anger and aggressive feelings and behavior—potentially directed towards the newborn, or partners and parents—and a variety of somatic symptoms that may also be present which may include varying degrees of fatigue, back pain, breast tenderness and discomfort, headaches and other manifestations—as well as underlying emotional and physical problems that may be exacerbated including dysphoria, anxiety and Post-Traumatic Stress Disorder, but not limited to these.

In a related aspect, the invention features a method of treating postpartum symptoms in a subject in need thereof, the method including administering to the subject ketamine, norketamine, 6-hydroxynorketamine, or a pharmaceutically acceptable salt thereof, in an amount sufficient to treat postpartum symptoms. In particular embodiments, the method ameliorates dysphoria, diminished interest or pleasure in activities, decrease or increase in appetite, troubled sleep or hypersomnia, insomnia, psychomotor agitation or retardation, fatigue or loss of energy, irritability, feelings of worthlessness or excessive or inappropriate guilt, diminished ability to think or concentrate or indecisiveness, inability to bond with the infant, anhedonia, anxiety, depression and in more severe states recurrent thoughts of death, suicidal ideation or suicidal attempts and anger and aggressive feelings and behavior—potentially directed towards the newborn, or partners and parents—and a variety of somatic symptoms that may also be present which may include varying degrees of fatigue, back pain, breast tenderness and discomfort, headaches and other manifestations—as well as underlying emotional and physical problems that may be exacerbated including dysphoria, anxiety and Post-Traumatic Stress Disorder, but not limited

In one embodiment of any of the above methods, the method can include initiating treatment following the appearance of a postpartum related symptom in the subject.

In any of the above methods the ketamine, norketamine, 6-hydroxynorketamine, or a pharmaceutically acceptable salt thereof, is administered once or more daily, once or more every other day, or once or more every three days, or depending on the presence and/or severity of postpartum related symptoms. For example, the can be once or twice daily for a period of from 1 to 10 days (e.g., for a period of from 2 to 8 days, from 2 to 7 days, from 2 to 6 days, or for a period of 2 days, 3 days, 4 days, 5 days, 6 days, 7 days, 8 days, 9 days, or 10 days), followed by a period of at least one or two weeks during which no ketamine, norketamine, 6-hydroxynorketamine, or a pharmaceutically acceptable salt thereof, is administered to the subject. In particular embodiments, the administration is once or more daily, or intermittently for a period of from 1 to 8 days, followed by a period of at least one or two weeks during which no ketamine, norketamine, 6-hydroxynorketamine, or a pharmaceutically acceptable salt thereof, is administered to the subject. In particular embodiments, the ketamine, norketamine, 6-hydroxynorketamine, or a pharmaceutically acceptable salt thereof, is administered to the subject only in the evening. In particular embodiments, the ketamine, norketamine, 6-hydroxynorketamine, or a pharmaceutically acceptable salt thereof, is administered to the subject once or more daily in the evening. In particular embodiments, the ketamine, norketamine, 6-hydroxynorketamine, or a pharmaceutically acceptable salt thereof, is administered to the subject during the daytime one or more times. In particular embodiments, the ketamine, norketamine, 6-hydroxynorketamine, or a pharmaceutically acceptable salt thereof, is administered to the subject one or more times daily and/or in the evening.

In a related aspect, the invention features a method of treating endometriosis in a subject in need thereof, the method including administering to the subject ketamine, norketamine, 6-hydroxynorketamine, or a pharmaceutically acceptable salt thereof, in an amount sufficient to treat the endometriosis. In particular embodiments, the method ameliorates emotional sensitivity, diminished memory, intellectual impairment, mood swings, dysphoria, anxiety, irritability, or diminished well-being in the subject; ameliorates a physical problem in the subject, such as headache, cramps, back pain, joint pain, or breast tenderness; and/or ameliorates hypersomnia, insomnia, difficulty in concentration, or lethargy in the subject. In one embodiment, the ketamine, norketamine, 6-hydroxynorketamine, or a pharmaceutically acceptable salt thereof, is administered once or more daily, once or more every other day, or once or more every three days, or once or more daily as needed to treat the symptoms of endometriosis. In particular embodiments, the ketamine, norketamine, 6-hydroxynorketamine, or a pharmaceutically acceptable salt thereof, is administered to the subject during the day and/or in the evening. In one particular embodiment, the endometriosis is refractory to treatment with NSAIDs, corticosteroids, muscle relaxants, or antidepressants

In particular embodiments of any of the above methods, the method includes administering an average daily dose of from 1 mg to 500 mg (e.g., 10 mg to 200 mg, 25 mg to 150 mg, or 35 mg to 125 mg) of ketamine, or a pharmaceutically acceptable salt thereof, to the subject. For example, the method can include administering an average daily dose of from 10 mg to 200 mg (e.g., 30±20 mg, 60±20 mg, 90±20 mg, or 150±50 mg) of enantiomerically pure S-(+)-ketamine, or a pharmaceutically acceptable salt thereof, to the subject. In particular embodiments, the method includes administering an average daily dose of from 10 mg to 200 mg (e.g., 30±20 mg, 60±20 mg, 90±20 mg, or 150±50 mg) of enantiomerically pure R-(−)-ketamine, or a pharmaceutically acceptable salt thereof, to the subject. In some embodiments, the method includes administering an average daily dose of from 10 mg to 200 mg (e.g., 30±20 mg, 60±20 mg, 90±20 mg, or 150±50 mg) of racemic ketamine, or a pharmaceutically acceptable salt thereof, to the subject.

In some embodiments of any of the above methods, the method includes administering one or more doses of from 1 mg to 500 mg (e.g., 10 mg to 200 mg, 25 mg to 150 mg, or 35 mg to 125 mg) of norketamine, or a pharmaceutically acceptable salt thereof, to the subject. For example, the method can include administering an average daily dose of from 10 mg to 200 mg (e.g., 30±20 mg, 60±20 mg, 90±20 mg, or 150±50 mg) of enantiomerically pure S-(+)-norketamine, or a pharmaceutically acceptable salt thereof, to the subject. In particular embodiments, the method includes administering an average daily dose of from 10 mg to 200 mg (e.g., 30±20 mg, 60±20 mg, 90±20 mg, or 150±50 mg) of enantiomerically pure R-(−)-norketamine, or a pharmaceutically acceptable salt thereof, to the subject. In some embodiments, the method includes administering an average daily dose of from 10 mg to 200 mg (e.g., 30±20 mg, 60±20 mg, 90±20 mg, or 150±50 mg) of racemic norketamine, or a pharmaceutically acceptable salt thereof, to the subject.

In some embodiments of any of the above methods, the method includes administering one or more doses of from 1 mg to 500 mg (e.g., 10 mg to 200 mg, 25 mg to 150 mg, or 35 mg to 125 mg) of 6-hydoxynorketamine, or a pharmaceutically acceptable salt thereof, to the subject. For example, the method can include administering an average daily dose of from 10 mg to 200 mg (e.g., 30±20 mg, 60±20 mg, 90±20 mg, or 150±50 mg) of isomerically pure (2R,6R)-6-hydroxynorketamine, or a pharmaceutically acceptable salt thereof, to the subject. In particular embodiments, the method includes administering an average daily dose of from 10 mg to 200 mg (e.g., 30±20 mg, 60±20 mg, 90±20 mg, or 150±50 mg) isomerically pure (2S,6S)-6-hydroxynorketamine, or a pharmaceutically acceptable salt thereof, to the subject.

In particular embodiments of any of the above methods, the administration is by a route selected from oral, sublingual, intranasal, intramuscular, intravenous, transdermal, vaginal, and rectal administration, or any administration route described herein.

In particular embodiments of any of the above methods, the ketamine, norketamine, 6-hydroxynorketamine, or a pharmaceutically acceptable salt thereof, is administered in an amount, or in a dosage form (e.g., a sustained release dosage form), that, upon administration to the subject, does not result in anesthesia in the subject.

The methods of the invention can include concurrent use of a second therapeutic agent.

In some embodiments, the method further includes concurrently administering to the subject a muscle relaxant. The muscle relaxant can be selected from afloqualone, baclofen, carisoprodol, chlormezanone, chlorphenesin carbamate, chlorzoxasozone, cyclobenzaprine, clonazepam, dantrolene, diazepam, eperisone, idrocilamide, inaperisone, mephenesin, mephenoxalone, methocarbamol, metaxalone, mivacurium chloride, orphenadrine, phenprobamate, pridinol mesylate, quinine, tetrazepam, thiocolchicoside, tizanidine, tolperisone, and pharmaceutically acceptable salts thereof.

In some embodiments, the method further includes concurrently administering to the subject an anti-inflammatory agent, such as NSAIDs, including ibuprofen in its various forms and naproxen in its various forms, and corticosteroids.

In some embodiments, the method further includes concurrently administering to the subject fluoxetine, or other anti-depressants described herein.

In certain embodiments of any of the above methods, the subject being treated is suffering from Postpartum Mood and Anxiety Disorders (PMAD), Postpartum Psychosis (PPP), Postpartum Depression (PPD), Anxiety (PPA), Post-Traumatic Stress (PPPTSD), Obsessive Compulsive Disorder (PPOCD), or Postpartum Bipolar I and II disorders (PPBPD).

In an embodiment of any of the above methods, the subject is breast feeding (e.g., a lactating mother). In certain embodiments, the following the administering, the subject does not express breast milk for consumption by a child for a period of at least 6 hours, 12 hours, 18 hours, or 24 hours, e.g., to reduce exposure of a breast feeding child to ketamine and/or ketamine metabolites in the breast milk. After at least 6 hours have passed after receiving ketamine, breast fed infants will be exposed to only minimal amounts of ketamine and its active metabolite norketamine. Breast milk can be discarded through 6 hours, 12 hours, 18 hours, or 24 hours, and breast feeding resumed thereafter.

As used herein, the term “average daily dose” refers to the average amount of ketamine, norketamine, 6-hydroxynorketamine, or a pharmaceutically acceptable salt thereof, administered to a subject for a given dosing regimen. For example, single doses of 100 mg of ketamine administered every other day is an average daily dose of 50 mg. For a regimen in which 15 mg doses of ketamine are administered twice daily, the average daily dose is 30 mg.

By “corticosteroid” is meant any naturally occurring or synthetic compound characterized by a hydrogenated cyclopentanoperhydrophenanthrene ring system. Naturally occurring corticosteroids are generally produced by the adrenal cortex. Synthetic corticosteroids may be halogenated. Exemplary corticosteroids are described herein.

As used herein, the term “pharmaceutically acceptable salts” refers to salts of the active compounds of the invention that can be prepared in situ during the final isolation and purification of the compounds of the invention, or separately by reacting the free base function with a suitable organic or inorganic acid. Representative acid addition salts include acetate, adipate, alginate, ascorbate, aspartate, benzenesulfonate, benzoate, bisulfate, camphorate, camphersulfonate, citrate, ethanesulfonate, hydrobromide, hydrochloride, hydroiodide, 2-hydroxy-ethanesulfonate, lactate, malate, maleate, malonate, mesylate, methanesulfonate, oxalate, succinate, sulfate, and tartrate salts.

A “therapeutically effective amount” or “an amount sufficient” of a drug is an amount effective to demonstrate a desired activity of the drug. According to the instant invention, a therapeutically effective amount of ketamine is an amount effective to alleviate, i.e., noticeably reduce, the symptoms of postpartum related symptoms.

As used herein, the term “treating” refers to administering a pharmaceutical composition for therapeutic purposes. To “treat disease” or use for “therapeutic treatment” refers to administering treatment to a subject already suffering from a condition to improve or stabilize the subject's condition.

### DETAILED DESCRIPTION

The invention for the first time provides a method of treating postpartum related symptoms. The compositions of the invention may be designed to be short-acting, fast-releasing, long-acting, or sustained-releasing as described herein. Thus, the pharmaceutical formulations may also be formulated for controlled release or for slow release.

Ketamine is an inexpensive, readily available drug, with minor adverse side effects. Thus, the invention contemplates additional savings to the overburdened health care system. Sublingual administration of this agent is rapid, allowing for fast action of the drug, and readily accomplished by a non-medically trained practitioner under medical supervision; by an MD or other medically licensed practitioner; and may be safely administered under close supervision in an at-home program. Intramuscular (IM) administration is a rapid acting method for ameliorating the symptoms as described herein. No other methods of administration are excluded from the Invention.

Contemplated herein is a package comprising a carrier for delivering a sublingual lozenge (troche) or oral dissolving tablets containing ketamine. Oral (buccal, sublingual) mucosal absorption of ketamine is a reliable method for administering ketamine that poses little hazard for abuse. Using sub-anesthetic dosages at specific levels designed to provide relief of postpartum related symptoms is the goal of this and any other methods, such as the possibility of an intranasal formulation for administering ketamine on its own or coupled with other agents for this indication.

A further advantage of the invention when provided for at-home use is that the patient can administer ketamine on an as needed, dose-to-effect basis. Thus, the frequency of administration is under the partial control of the patient, with close medical supervision. However, the relatively low dose with each administration and the sublingual route of administration will reduce the possibilities for abuse, especially since it is difficult to use multiple lozenges at the same time. Yet another particular advantage of the present invention is that sublingual administration of ketamine is non-invasive, and provides for rapid introduction of effect within minutes. Control of the frequency and quantity of prescription is entirely under the physician's medical judgement.

Ketamine's many decades long use has established its safety for human use at dosages far in excess of those being contemplated for this application. However, it remains to demonstrate the pharmacokinetics of ketamine in lactation. While there are statements as to its safety in infants who are being breast-fed, these remain unsupported by actual scientific evaluation. Attendant to this patent and use as indicated, a study that will illuminate the profile of ketamine's presence in breast milk will be undertaken. It is the intention of this work to provide breast-feeding women with an awareness of the actual presence of ketamine in breast milk. While there will be no possibility of assessing its safety for infants, our intent is ,minimize exposure to ketamine for infants who are being breast-fed.

Ketamine's application in a psychotherapeutic process is essential for the success of treating postpartum symptoms and disorders. Women need support, a sense of being held in a therapeutic nest with involvement of their partners and support systems While ketamine employed as a drug in a medicalized setting may well have some benefit—and is not excluded within the framework of this invention—it is not the preferred method of ketamine's administration for the spectrum of postpartum symptoms and disorders.

As discussed above, the present invention is directed to various methods and compositions for treating postpartum related symptoms. An alternative route of administration could comprise intranasal administration of ketamine. While the sublingual route is preferred because of its lower possibility for abuse, an intranasal preparation for this method may be contemplated with appropriate safeguards for administration. Such treatment may be administered alone or may be supplemented with other therapies as described herein. An IM method of administration may well be the principle method used in this treatment of postpartum related symptoms with the sub-lingual method as supportive and/or primary.

### Ketamine and Norketamine

The methods of the invention include the administration of ketamine, norketamine, 6-hydroxynorketamine, or a pharmaceutically acceptable salt thereof.

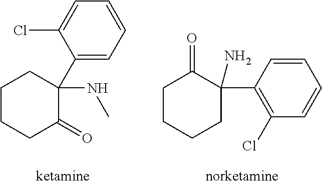 [SMILES: CNC1(c2ccccc2Cl)CCCCC1=O ; NC1(c2ccccc2Cl)CCCCC1=O]

As used herein, the term “ketamine” includes ketamine in its racemic (R/S) form, in its R-(−) enantiomerically pure form, or in its S-(+) enantiomerically pure form.

As used herein, the term “norketamine” includes norketamine in its racemic (R/S) form, in its R-(−) enantiomerically pure form, or in its S-(+) enantiomerically pure form.

As used herein, “enantiomerically pure” refers to compositions consisting substantially of a single isomer (i.e., substantially free of the opposite isomer), preferably consisting of 90%, 92%, 95%, 98%, 99%, or 100% (w/w) of a single isomer. For example, when the methods of the invention include the administration of enantiomerically pure R-(−)-ketamine, the pharmaceutical composition administered can include at least 95% (w/w) S-(+)-ketamine, and less than 5% (w/w) R-(−)-ketamine.

Ketamine racemate is primarily used for the induction and maintenance of general anesthesia. Enantiomerically pure S-(+)-ketamine (aka esketamine) is available for medical use, administered either IV (intravenously) or IM (intramuscularly), under the brand name KETANEST®. Enantiomerically pure R-(−)-ketamine is also known as arketamine. Ketamine is converted metabolically through demethylation to norketamine, in vivo, at rates dependent on the route of administration. For use in anesthesia, S-(+)-ketamine has been reported to be twice as potent as R-(−)-ketamine, and norketamine has been reported to have one third the potency of ketamine (C. S. T. Aun, Br. J. Anaesthesia 83: 29-41 (1999)).

As used herein, the term “6-hydroxynorketamine” includes 6-hydroxynorketamine in any of its 2R,6R; 2S,6S; 2S,6R; and 2R,6S isomerically pure forms (shown below).

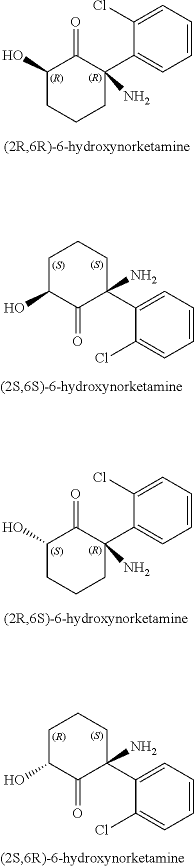

As used herein, “isomerically pure” refers to compositions consisting substantially of a single diastereomer (i.e., substantially free of other isomers), preferably consisting of 90%, 92%, 95%, 98%, 99%, or 100% (w/w) of a single isomer. For example, when the methods of the invention include the administration of isomerically pure (2R,6R)-6-hydroxynorketamine, the pharmaceutical composition administered can include at least 95% (w/w) (2R,6R)-6-hydroxynorketamine, and less than 5% (w/w) other isomers of 6-hydroxynorketamine. Alternatively, the methods of the invention include the administration of isomerically pure (2S,6S)-6-hydroxynorketamine, the pharmaceutical composition administered can include at least 95% (w/w) (2S,6S)-6-hydroxynorketamine, and less than 5% (w/w) other isomers of 6-hydroxynorketamine.

### Formulation of Pharmaceutical Compositions

The administration of ketamine, norketamine, 6-hydroxynorketamine, or a pharmaceutically acceptable salt thereof, (the active compound) may be by any suitable means that results in relief of a postpartum conditions and symptoms. The active may be contained in any appropriate amount in any suitable carrier substance, and is generally present in an amount of 1-95% by weight of the total weight of the composition. The composition may be provided in a dosage form that is suitable for the sublingual, buccal, oral, parenteral (e.g., intravenously, intramuscularly), intranasal, transdermal, vaginal, or rectal administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, sprays, or aerosols. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the active compound within the body over an extended period of time; (ii) formulations that after a predetermined lag time create a substantially constant concentration of the active compound within the body over an extended period of time; and (iii) formulations that sustain active compound action during a predetermined time period by maintaining a relatively, constant, effective active compound level in the body with concomitant minimization of undesirable side effects associated with fluctuations in the plasma level of the active compound (sawtooth kinetic pattern).

Administration of the active compound in the form of a controlled release formulation is especially preferred in cases in which the active compound, either alone or in combination with a second agent, at therapeutic levels produces unwanted side effects, such as nausea.

Any of a number of strategies can be pursued in order to obtain controlled release of the active compound in question. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the active compound in a controlled manner. Examples include single or multiple unit tablet or capsule compositions, oil solutions, suspensions, emulsions, microcapsules, microspheres, nanoparticles, patches, and liposomes.

Sublingual and Buccal Dosage Forms

Formulations for sublingual may be in the form of films, strips, lozenges, and orally dissolving tablets. An orally dissolving tablet (ODT) refers to pharmaceutical dosage form designed to be dissolved on the tongue rather than swallowed whole, or designed to dissolve on the sublingual or buccal mucosa for sublingual or mucosal administration. Alternatively, the dosage form can be a lozenge (for slower administration as the lozenge dissolved over the course of 5-10 minutes), or as a rapidly dissolving film (dissolving over the course of less than 2 minutes). The active compound is administered via absorption in the mouth (i.e., buccally or sublingually). The formulation excipients are edible and pharmaceutically acceptable using excipients known in the art for the preparation of films, strips, lozenges, and orally dissolving tablets. For example, a film is prepared typically using hydrophilic polymers that rapidly dissolves on the tongue, palatine tissue, or buccal cavity, delivering the active compound to the systemic circulation via dissolution when contact with liquid is made.

Solid Dosage Forms for Oral Use

Formulations for oral use include tablets containing the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active compound in a predetermined pattern (e.g., in order to achieve a controlled release formulation) or it may be adapted not to release the active compound until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active compound). The coating may be applied on the solid dosage form in a similar manner as that described in Encyclopedia of Pharmaceutical Technology, supra.

For combination therapies, two drugs may be mixed together in the tablet, or may be partitioned. In one example, the first drug is contained on the inside of the tablet, and the second drug is on the outside, such that a substantial portion of the second drug is released prior to the release of the first drug.

Formulations for oral use may also be presented as chewable tablets, or as hard gelatin capsules wherein the active compound is mixed with an inert solid diluent (e.g., potato starch, lactose, microcrystalline cellulose, calcium carbonate, calcium phosphate or kaolin), or as soft gelatin capsules wherein the active compound is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active compound by controlling the dissolution and/or the diffusion of the active drug substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more of the compounds of the claimed combinations may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the drug(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Liquids for Oral Administration

Powders, dispersible powders, or granules suitable for preparation of an aqueous suspension by addition of water are convenient dosage forms for oral administration. Formulation as a suspension provides the active compound in a mixture with a dispersing or wetting agent, suspending agent, and one or more preservatives. Suitable dispersing or wetting agents are, for example, naturally-occurring phosphatides (e.g., lecithin or condensation products of ethylene oxide with a fatty acid, a long chain aliphatic alcohol, or a partial ester derived from fatty acids) and a hexitol or a hexitol anhydride (e.g., polyoxyethylene stearate, polyoxyethylene sorbitol monooleate, polyoxyethylene sorbitan monooleate, and the like). Suitable suspending agents are, for example, sodium carboxymethylcellulose, methylcellulose, sodium alginate, and the like.

Parenteral Compositions

The pharmaceutical composition may also be administered parenterally by injection, infusion or implantation (intravenous, intramuscular, subcutaneous, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active compound, the composition may include suitable parenterally acceptable carriers and/or excipients. The active drug(s) may be incorporated into microspheres, microcapsules, nanoparticles, liposomes, or the like for controlled release. Furthermore, the composition may include suspending, solubilizing, stabilizing, pH-adjusting agents, and/or dispersing agents.

As indicated above, the pharmaceutical compositions according to the invention may be in the form suitable for sterile injection. To prepare such a composition, the suitable active compound is dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution. The aqueous formulation may also contain one or more preservatives (e.g., methyl, ethyl or n-propyl p-hydroxybenzoate).

Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug(s) may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters)).

Rectal Compositions

For rectal application, suitable dosage forms for a composition include suppositories (emulsion or suspension type), and rectal gelatin capsules (solutions or suspensions). In a typical suppository formulation, the active compound is combined with an appropriate pharmaceutically acceptable suppository base such as cocoa butter, esterified fatty acids, glycerinated gelatin, and various water-soluble or dispersible bases like polyethylene glycols and polvoxyethylene sorbitan fatty acid esters. Various additives, enhancers, or surfactants may be incorporated.

Vaginal Compositions

For vaginal application, suitable dosage forms for a composition include suppositories (emulsion or suspension type), and vaginal gelatin capsules (solutions or suspensions). In a typical suppository formulation, the active compound is combined with an appropriate pharmaceutically acceptable suppository base such as cocoa butter, esterified fatty acids, glycerinated gelatin, and various water-soluble or dispersible bases like polyethylene glycols and polvoxyethylene sorbitan fatty acid esters. Various additives, enhancers, or surfactants may be incorporated.

Intranasal and Inhalation Compositions

For administration by inhalation, typical dosage forms include nasal sprays and aerosols. In a typically nasal formulation, the active compound is dissolved or dispersed in a suitable vehicle. The pharmaceutically acceptable vehicles and excipients (as well as other pharmaceutically acceptable materials present in the composition such as diluents, enhancers, flavoring agents, and preservatives) are selected in accordance with conventional pharmaceutical practice in a manner understood by the persons skilled in the art of formulating pharmaceuticals.

Percutaneous and Topical Compositions

The pharmaceutical compositions may also be administered topically on the skin for percutaneous absorption in dosage forms or formulations containing conventionally non-toxic pharmaceutical acceptable carriers and excipients including microspheres and liposomes. The formulations include creams, ointments, lotions, liniments, gels, hydrogels, solutions, suspensions, sticks, sprays, pastes, plasters, and other kinds of transdermal drug delivery systems. The pharmaceutically acceptable carriers or excipients may include emulsifying agents, antioxidants, buffering agents, preservatives, humectants, penetration enhancers, chelating agents, gel-forming agents, ointment bases, perfumes, and skin protective agents.

Examples of emulsifying agents are naturally occurring gums (e.g., gum acacia or gum tragacanth) and naturally occurring phosphatides (e.g., soybean lecithin and sorbitan monooleate derivatives). Examples of antioxidants are butylated hydroxy anisole (BHA), ascorbic acid and derivatives thereof, tocopherol and derivatives thereof, butylated hydroxy anisole, and cysteine. Examples of preservatives are parabens, such as methyl or propyl p-hydroxybenzoate, and benzalkonium chloride. Examples of humectants are glycerin, propylene glycol, sorbitol, and urea. Examples of penetration enhancers are propylene glycol, DMSO, triethanolamine, N,N-dimethylacetamide, N,N-dimethylformamide, 2-pyrrolidone and derivatives thereof, tetrahydrofurfuryl alcohol, and AZONE™. Examples of chelating agents are sodium EDTA, citric acid, and phosphoric acid. Examples of gel forming agents are CARBOPOL™, cellulose derivatives, bentonite, alginates, gelatin and polyvinylpyrrolidone. Examples of ointment bases are beeswax, paraffin, cetyl palmitate, vegetable oils, sorbitan esters of fatty acids (Span), polyethylene glycols, and condensation products between sorbitan esters of fatty acids and ethylene oxide (e.g., polyoxyethylene sorbitan monooleate (TWEEN™)).

The compositions may be adapted for direct application or for introduction into relevant orifice(s) of the body (e.g., rectal, urethral, vaginal or oral orifices). The composition may be applied by means of special drug delivery devices such as dressings or alternatively plasters, pads, sponges, strips, or other forms of suitable flexible material.

Dosages

The dosage of ketamine, norketamine, 6-hydroxynorketamine, or a pharmaceutically acceptable salt thereof, to be administered depends on several factors, including: the administration method, the condition or symptom to be treated, the severity of the condition or symptom, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect dosage used.

As described above, the active compound may be administered orally in the form of tablets, capsules, elixirs or syrups; or vaginally or rectally in the form of suppositories. Parenteral administration of the active compound is suitably performed, for example, in the form of saline solutions or with the compound incorporated into liposomes. Sublingual or buccal administration of the active compound may be in the form of films, strips, lozenges, and orally dissolving tablets.

The amount administered can be from about 0.01 mg of active compound per kg of the subject's body weight (mg/kg) to about 5 mg/kg (e.g., from about 0.05 mg/kg to about 1 mg/kg, from about 0.05 mg/kg to about 0.5 mg/kg, from about 1 mg/kg to about 3 mg/kg, or from about 2 mg/kg to about 5 mg/kg), depending upon the route of administration. In general, the dose will be in the range of about 10 mg/day to about 200 mg/day (e.g., from about 25 mg/day to about 150 mg/day or from about 50 mg/day to about 200 mg/day) for sublingual administration. The dose will be in the range of about 5 mg/day to about 100 mg/day (e.g., from about 5 mg/day to about 50 mg/day or from about 25 mg/day to about 100 mg/day) for intranasal administration. The dose will be in the range of about 2 mg/day to about 75 mg/day (e.g., from about 2 mg/day to about 35 mg/day or from about 25 mg/day to about 75 mg/day) for intravenous administration. The dose will be in the range of about 10 mg/day to about 75 mg/day (e.g., from about 10 mg/day to about 45 mg/day or from about 40 mg/day to about 75 mg/day) for intramuscular administration. The dose will be in the range of about 50 mg/day to about 250 mg/day (e.g., from about 50 mg/day to about 125 mg/day or from about 100 mg/day to about 250 mg/day) for transdermal administration.

Therapy

Therapy according to the invention may be provided at home, the doctor's office, a clinic, a hospital's outpatient department, or a hospital. Treatment begins at the physician's office so that the doctor can observe the therapy's effects closely and make any adjustments that are needed. The duration of the therapy depends on the type and severity of the postpartum related symptoms being treated, the age and condition of the patient, the stage and type of the patient's postpartum related condition, and how the patient responds to the treatment.

Optionally, the ketamine, norketamine, 6-hydroxynorketamine, or a pharmaceutically acceptable salt thereof, is administered in combination with a second agent (e.g., a muscle relaxant or an anti-inflammatory agent). For combination therapies, the dosage, frequency and mode of administration of each component of the combination can be controlled independently. For example, ketamine, norketamine, 6-hydroxynorketamine, or a pharmaceutically acceptable salt thereof, may be administered sublingually in a regimen described herein, while the second agent may be administered orally once per day. The combination of therapeutic agents may also be formulated together such that one administration delivers both actives.

Muscle Relaxants

If desired, the ketamine, norketamine, 6-hydroxynorketamine, or a pharmaceutically acceptable salt thereof, may be administered in conjunction with one or more muscle relaxants, such as afloqualone, baclofen, carisoprodol, chlormezanone, chlorphenesin carbamate, chlorzoxasozone, cyclobenzaprine, clonazepam, dantrolene, diazepam, eperisone, idrocilamide, inaperisone, mephenesin, mephenoxalone, methocarbamol, metaxalone, mivacurium chloride, orphenadrine, phenprobamate, pridinol mesylate, quinine, tetrazepam, thiocolchicoside, tizanidine, tolperisone, and pharmaceutically acceptable salts thereof. Two or more muscle relaxants can be administered in the same treatment. This combination can be especially useful for situations in which the dominant postpartum related symptoms experienced by the subject include cramping.

Non-Steroidal Anti-Inflammatory Drugs (NSAIDs)

If desired, the ketamine, norketamine, 6-hydroxynorketamine, or a pharmaceutically acceptable salt thereof, may be administered in conjunction with one or more non-steroidal anti-inflammatory drugs (NSAIDs), such as naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin. Two or more NSAIDs can be administered in the same treatment. This combination can be especially useful for situations in which the dominant postpartum related symptom experienced by the subject is physical pain.

Corticosteroids

If desired, the ketamine, norketamine, 6-hydroxynorketamine, or a pharmaceutically acceptable salt thereof, may be administered in conjunction with one or more corticosteroids. Suitable corticosteroids include 11-alpha,17-alpha,21-trihydroxypregn-4-ene-3,20-dione; 11-beta,16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclometasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; dihydroelatericin a; domoprednate; doxibetasol; ecdysone; ecdysterone; endrysone; enoxolone; flucinolone; fludrocortisone; fludrocortisone acetate; flugestone; flumethasone; flumethasone pivalate; flumoxonide; flunisolide; fluocinolone; fluocinolone acetonide; fluocinonide; 9-fluorocortisone; fluocortolone; fluorohydroxyandrostenedione; fluorometholone; fluorometholone acetate; fluoxymesterone; fluprednidene; fluprednisolone; flurandrenolide; fluticasone; fluticasone propionate; formebolone; formestane; formocortal; gestonorone; glyderinine; halcinonide; hyrcanoside; halometasone; halopredone; haloprogesterone; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone diacetate; triamcinolone hexacetonide; trimegestone; turkesterone; and wortmannin.

Standard recommended dosages for corticosteroids are provided, e.g., in the Merck Manual of Diagnosis & Therapy (17th Ed. MH Beers et al., Merck & Co.) and Physicians' Desk Reference 2003 (57th Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone Two or more corticosteroids can be administered in the same treatment. This combination can be especially useful for situations in which the dominant postpartum related symptom experienced by the subject is swelling.

Antidepressants

If desired, the ketamine, norketamine, 6-hydroxynorketamine, or a pharmaceutically acceptable salt thereof, may be administered in conjunction with one or more antidepressants, such as fluoxetine, duloxetine, bupropion, citalopram, escitalopram, paroxetine, lorazepam, fluvoxamine, sertraline, desvenlafaxine, milnacipran, venlafaxine, amitriptyline, nortriptyline, desipramine, alprazolam, agomelatine, etoperidone, or phenelzine. Two or more antidepressants can be administered in the same treatment. This combination can be especially useful for situations in which the dominant postpartum related symptom experienced by the subject is anxiety or dysphoria.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1

### Pharmacodynamics of Ketamine in the Breast Milk of Lactating Women

A study of the pharmacodynamics of ketamine in the breast milk of lactating women was performed. Ketamine levels in breast milk were quantified in lactating subjects over time. Four women were given intramuscular injections of ketamine hydrochloride at two doses: 0.5 mg/kg and 1.0 mg/kg at intervals of at least one week apart. Breast milk was collected in one subject at 3 hour intervals through 24 hours, and the succeeding three subjects at 3 hour intervals up to 12 hours. Subjects were of different weights and ethnic backgrounds.

Administered dosages of ketamine ranged from 28 mg to 76 mg. The milk samples were analyzed for the presence of ketamine, the active metabolite norketamine (estimated to be one-third the potency of ketamine) and two inert metabolites. For subjects receiving the 0.5 mg/kg dose, the observed range for ketamine in the breast milk of the subject at the 9 to 12 hour collection period (total expression of milk during the period) ranged from 0.09 mg to 1.66 mg. For subjects receiving the 1.0 mg/kg dose, the observed range for ketamine in the breast milk of the subject at the 9 to 12 hour collection period (total expression of milk during the period) ranged from 1.067 mg to 6.22 mg. The highest ketamine level (6.22 mg) was observed following administration of a 76 mg dose of ketamine hydrochloride. The results were consistent in demonstrating that at 12 hours only minimal levels of ketamine and its active metabolite norketamine were detectable.

These results show that in lactating women that treated for PPD withholding breast feeding for 12 hours following ketamine administration in any format will deliver only minimal amounts of it to infants who are breast feeding. This study supports the use of ketamine assisted psychotherapy (KAP) in postpartum depression and other postpartum emotional disorders with only a brief interruption of breast feeding necessary to prevent exposure to infants (e.g., at least 6 hours, 12 hours, 18 hours, or 24 hours) and maintains the integrity of the relationship between mother and child. As conventional treatment of postpartum emotional disorders relies on continuous administration of antidepressants and other medications, KAP offers both highly effective treatment for depression and other emotional disorders, and in this application does not expose infants to the risks associated with a continuous influx of drugs that may well effect current behavior, and possibly longer term, development.

### OTHER EMBODIMENTS

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

What is claimed is:

1. A method of treating a postpartum related symptom in a subject in need thereof, the method comprising administering to the subject ketamine, norketamine, 6-hydroxynorketamine, or a pharmaceutically acceptable salt thereof, in an amount sufficient to treat the postpartum related symptom.

2. The method of claim 1, wherein the postpartum related symptom is selected from dysphoria, diminished interest or pleasure in activities, decrease or increase in appetite, troubled sleep or hypersomnia, insomnia, psychomotor agitation or retardation, fatigue or loss of energy, irritability, feelings of worthlessness or excessive or inappropriate guilt, diminished ability to think or concentrate or indecisiveness, inability to bond with the infant, anhedonia, anxiety, depression and in more severe states recurrent thoughts of death, suicidal ideation or suicidal attempts and anger and aggressive feelings and behavior—potentially directed towards the newborn, or partners and parents—and a variety of somatic symptoms that may also be present which may include varying degrees of fatigue, back pain, breast tenderness and discomfort, headaches and other manifestations—as well as underlying emotional and physical problems that may be exacerbated including dysphoria, anxiety and Post-Traumatic Stress Disorder, but not limited to these.

3. The method of claim 1, wherein the postpartum related symptom is hypersomnia, insomnia, difficulty in concentration, or lethargy.

4. The method of any one of claims 1-2 wherein the postpartum related symptom is refractory to treatment with NSAIDs, corticosteroids, muscle relaxants, antidepressants or other psychiatric or alternative medications.

5. The method of any one of claims 1-4, wherein the method comprises initiating treatment following the appearance of a postpartum related symptom in the subject.

6. The method of claims 1-5, wherein the ketamine, norketamine, 6-hydroxynorketamine, or a pharmaceutically acceptable salt thereof, is administered once or more daily, once or more every other day, or once or more every three days, depending on the presence and/or severity of postpartum related symptoms.

7. The method of any one of claims 1-5, wherein the administration is once daily for a period of from 1 to 10 days, followed by a period of at least two weeks during which no ketamine, norketamine, 6-hydroxynorketamine, or a pharmaceutically acceptable salt thereof, is administered to the subject.

8. The method of claims 1-5, wherein the administration is once daily for a period of from 1 to 8 days, followed by a period of at least two weeks during which no ketamine, norketamine, 6-hydroxynorketamine, or a pharmaceutically acceptable salt thereof, is administered to the subject.

9. The method of any one of claims 1-5 wherein the ketamine, norketamine, 6-hydroxynorketamine, or a pharmaceutically acceptable salt thereof, is administered to the subject once or more daily and/or in the evening. Or in a program of frequency related to the needs of the patient for relief from postpartum related symptoms.

10. The method of claims 1-5, wherein the method comprises administering an average daily dose of from 10 mg to 200 mg of racemic ketamine, or a pharmaceutically acceptable salt thereof, to the subject.

11. The method of any one of claims 1-5, wherein the method comprises administering one or more doses of from 1 mg to 200 mg of norketamine, or a pharmaceutically acceptable salt thereof, to the subject.

12. The method of claims 1-5, wherein the method comprises administering an average daily dose of from 10 mg to 200 mg of enantiomerically pure S-(+)-norketamine, or a pharmaceutically acceptable salt thereof, to the subject.

13. The method of claims 1-5, wherein the method comprises administering an average daily dose of from 10 mg to 200 mg of enantiomerically pure R-(−)-norketamine, or a pharmaceutically acceptable salt thereof, to the subject.

14. The method of claims 1-5, wherein the method comprises administering an average daily dose of from 10 mg to 200 mg of racemic norketamine, or a pharmaceutically acceptable salt thereof, to the subject.

15. The method of any one of claims 1-5, wherein the method comprises administering one or more doses of from 1 mg to 200 mg of 6-hydoxynorketamine, or a pharmaceutically acceptable salt thereof, to the subject.

16. The method of claims 1-5, wherein the method comprises administering an average daily dose of from 10 mg to 200 mg of isomerically pure (2R,6R)-6-hydroxynorketamine, or a pharmaceutically acceptable salt thereof, to the subject.

17. The method of claims 1-5, wherein the method comprises administering an average daily dose of from 10 mg to 200 mg of isomerically pure (2S,6S)-6-hydroxynorketamine, or a pharmaceutically acceptable salt thereof, to the subject.

18. The method of any one of claims 1-5, wherein the administration is by a route selected from oral, sublingual, intranasal, intramuscular, intravenous, transdermal, vaginal, and rectal administration.

19. The method of any one of claims 1-5, further comprising concurrently administering to the subject a muscle relaxant.

20. The method of claim 19, wherein the muscle relaxant is selected from afloqualone, baclofen, carisoprodol, chlormezanone, chlorphenesin carbamate, chlorzoxasozone, cyclobenzaprine, clonazepam, dantrolene, diazepam, eperisone, idrocilamide, inaperisone, mephenesin, mephenoxalone, methocarbamol, metaxalone, mivacurium chloride, orphenadrine, phenprobamate, pridinol mesylate, quinine, tetrazepam, thiocolchicoside, tizanidine, tolperisone, and pharmaceutically acceptable salts thereof.

21. The method of any one of claims 1-5, further comprising concurrently administering to the subject an anti-inflammatory agent.

22. The method of claim 21, wherein the anti-inflammatory agent is selected from NSAIDs and corticosteroids.

23. The method of any one of claims 1-5, further comprising concurrently administering to the subject an antidepressant, or other psychiatric, or alternative medicine.

24. The method of any one of claims 1-5, wherein the subject is suffering from Postpartum Mood and Anxiety Disorders (PMAD), Postpartum Psychosis (PPP), Postpartum Depression (PPD), Anxiety (PPA), Post-Traumatic Stress (PPPTSD), Obsessive Compulsive Disorder (PPOCD), or Postpartum Bipolar I and II disorders (PPBPD).

25. The method of any one of claims 1-24, wherein the subject is breast feeding.

26. The method of claim 25, wherein following the administering, the subject does not express breast milk for consumption by a child for a period of at least 6 hours.
